(19) United States
(12) Patent Application Publication
(10) Pub. No.: US 2025/0135115 A1
(43) Pub. Date: May 1, 2025

(54) Arrangement for a drug delivery device, drug delivery device and user interface module

(71) Applicant: Sanofi, PARIS (FR)

(72) Inventor: Ronald Antony Smith, Salir, Loule (PT)

(21) Appl. No.: 18/694,096

(22) PCT Filed: Sep. 22, 2022
(86) PCT No.: PCT/EP2022/076284

§ 371 Date: Mar. 21, 2024

(30) Foreign Application Priority Data
Sep. 24, 2021 (EP) 21315182.2

(51) Int. Cl. A61M 5/20

(52) U.S. Cl. CPC A61M 5/2033; A61M 2205/50

(57) ABSTRACT

An arrangement for a drug delivery device is described. The arrangement includes: at least one electronic circuit, an antenna for wireless communication, and a body. The antenna is operatively connected to the electronic circuit, the antenna is flexible, and the antenna is adapted to follow a contour defined by the body.

### CROSS REFERENCE TO RELATED APPLICATIONS

The present application is the national stage entry of International Patent Application No. PCT/EP2022/076284, filed on Sep. 22, 2022, and claims priority to Application No. EP 21315182.2, filed on Sep. 24, 2021, the disclosures of which are incorporated herein by reference.

### TECHNICAL FIELD

The disclosure relates to an arrangement for a drug delivery device, e.g. an autoinjector or a manually driven injection device. The drug may comprise insulin, a growth hormone or some other medicament. The arrangement may comprise an antenna for communication purposes and a body.

### BACKGROUND

Drug delivery devices utilizing antennas have inherent advantages. Thus, it is possible to transmit medical data that may be used for monitoring and/or diagnostic purposes.

### SUMMARY

The disclosure describes an arrangement for a drug delivery device, in which the arrangement includes an antenna. The arrangement would enable a compact drug and/or medicament delivery device and/or good usability and/or simple manufacturing. Furthermore, the disclosure describes a corresponding drug delivery device and a corresponding user interface module.

In some embodiments, the arrangement for a drug delivery device includes: at least one electronic circuit, an antenna for wireless communication, and a body. The antenna is operatively connected to the electronic circuit, e.g., during the operation of the antenna, the antenna is flexible, and the antenna is adapted to follow a contour defined by the body.

“Flexible” may mean that it is possible to deflect the antenna in some way, for instance to deform the antenna from a first plane conformation to a second curved conformation.

The antenna may be resiliently flexible, e.g. there may be a remaining elastic deflection force that is able to bring the antenna back into an original plane. However, a resilient characteristic is not necessary as long as the antenna is flexible, e.g. the antenna may be plastically deformable, e.g. bendable.

The electronic circuit may comprise at least one electronic element, preferably a plurality of electronic elements and/or at least one printed circuit board. At least one of the electronic elements may be an integrated electronic circuit (IC). The integrated circuit may be produced using lithography technologies for semiconducting devices, e.g. within minimum dimensions below one micrometer or below 100 nanometers. Examples for integrated circuits which may be comprised in the electronic circuit are a processor, e.g. a microprocessor or a microcontroller that may be comprise more peripheral components compared to a microprocessor, and/or a timer integrated circuit (IC), etc.

The printed circuit board may also be produced using lithography technologies. However, minimum dimensions may be more than 1 micrometer.

The antenna may be a radio frequency antenna, for instance for transmission of data with a frequency within the range of 0.1 MHZ (megahertz) to 60 GHz or of 1 GHz to 20 GHz. The antenna may be designed to transmit electromagnetic waves of a single carrier frequency or of several carrier frequencies, e.g. a fractal antenna.

The body may be a housing element or may be part of a housing element. Alternatively, the body may be part of an inner chassis that may be surrounded by an outer housing element. The body may be a first housing member, e.g. a first housing member that carries the electronic circuit. Alternatively, the body may be a second housing member, e.g. a main housing of a drug delivery device. In this case, a further body may carry and/or surround the electronic circuit.

The body may have the shape of a hollow cylinder. The body may comprise a distal end that is configured to be arranged closer to an injection opening of the drug delivery device relative to a proximal end of the body. A longitudinal axis of the body may extend from the proximal end to the distal end.

The term “contour” may refer to the outer contour, e.g. outer outline, an inner contour, e.g. inner outline, and/or to the curvature within the three-dimensional space. As the antenna is flexible, the antenna may be adapted to follow a contour defined by the body. This may allow the construction of a compact drug delivery device and/or simple attachment of the antenna to the drug delivery device.

Without being bound by theory, especially by antenna theory, the antenna may be easily arranged along the body in a manner which enables an enhancement of isotropic radiation characteristic, e.g. with regard to transmission (sending) and/or with regard to a receiving characteristic of the antenna. Alternatively, more anisotropic characteristics may be realized using the proposed arrangement as well.

A curvature of the antenna may follow a curvature that is defined by the body. Thus, it may be possible to wrap the antenna around the body, e.g. less than one time, one time, about one time or several times. Thus, the curvature of the body may simply be used in order to define the intended curvature of the antenna. The antenna may be arranged on an outside of the body or on an inside of the body, especially if a hollow body is used.

The antenna may be arranged on a flexible carrier substrate. The flexible carrier substrate may be part of a circuit board of the electronic circuit, preferably part of a multilayer circuit board. The circuit board may comprise at least one, at least two, at least three or at least four flexible carrier substrates. Flexible portion(s) of the carrier substrate(s) may be arranged around a central portion which may be more rigid compared to the flexible portion, e.g. because of more conductive material. The central portion may be referred to as a mainboard. The flexibility of the carrier substrate(s) may enable the flexibility of the antenna. Furthermore, the flexible carrier substrate may enable an integral connection of parts of the electronic circuit to the mainboard, e.g. without usage of further connecting elements, e.g. connectors, solder connection, etc.

The multilayer circuit board may be appropriate for a very compact arrangement of several electronic components not only on the mainboard but also in planes or at locations which are not positioned within the plane in which the mainboard is arranged.

The antenna may be a conductive track or may comprise at least one conductive track of the circuit board. Conductive track may mean that the conductive track has a width that is greater than the height or thickness of the conductive track, for instance by at least factor 3, 4 or 5 or by at least factor 10 or 20 but for instance less than factor 10000. The conductive track may have a small thickness. The conductive track may be electrically conductive, e.g. comprising a metal or consisting of a metal, for instance copper or consisting of copper. The conductive track may consist of or may comprise conductive ink as is used in printed electronics.

The flexible carrier substrate or a single layer of the flexible carrier substrate may have a small thickness in order to enable bending or deflection of the antenna. The thickness of the flexible carrier substrate may also be small in order to enable flexibility. The flexible carrier substrate may be electrically isolating, e.g. a polyimide or another material. The electrical conductivity of the substrate may be less than 10⁻⁸ S/cm (Siemens/centimeter). Thus, the substrate may comprise a non-conducting/isolating flexible dielectric substrate material.

Thus, the flexible carrier substrate may be a multilayer substrate comprising at least two layers, at least three layers or at least four carrier layers. All these carrier substrates may extend along the antenna. This may simplify manufacturing of the printed circuit board as all carrier layers may have the same contour, e.g. only one punching tool may be used. However, only one or only some of these carrier substrate layers may carry conductive tracks that form the antenna and/or that are arranged along the antenna. Alternatively, not all of the carrier substrate layers may be arranged along the antenna.

Each carrier substrate or at least one of the carrier substrates may carry only one layer with conductive tracks, e.g. it may be a single sided carrier substrate (only one conductive track layer on one side). Alternatively, each or at least one of the carrier substrates may be a two-sided carrier substrate (two conductive track layers, one conductive track layer on each side). Electrically conductive tracks of different layers may be connected by vias, e.g. through holes comprising electrically coated side faces and/or filled by an electrically conductive material. Isolating layers may be used between adjacent track layers, e.g. prepregs.

The term “printed” circuit board may be used because the circuit board looks like it is printed although printing technologies have not been used. Other technologies may be used, for instance photolithography in combination with etching of conductive layers, e.g. of copper layers.

The antenna may be a monopole antenna or a dipole antenna. Thus, the antenna may be a simple antenna which may be manufactured in a simple way.

The monopole antenna may have only one connection port and only one conductor. The radiation characteristic of the monopole antenna may be adapted by adapting the spatial conformation of the antenna, e.g. by forming a loop.

The dipole antenna may comprise two connection ports. An open dipole (elongated) antenna or a folded dipole antenna may be used. The open dipole antenna may consist of only two (monopole) conductors each connected to one of the two connecting ports. The folded dipole may consist of a conductor loop comprising three conductor sections. A longer one of the conductor sections runs parallel to the other two conductor sections. Each of the other conductor sections are shorter than the longer conductor section and has typically only half of the length of the longer conductor section. Each of the shorter conductor sections are connected to one respective end of the longer conductor section. The free ends of the shorter conductor sections form the respective connection ports of the folded dipole. The radiation characteristic of the open dipole antenna or of the folded dipole antenna may also be adapted by adapting the conformation, e.g. by arranging the dipole along a loop.

The antenna, preferably a conductive track of the antenna, may have a length within the range of 1.5 cm to 5 cm (centimeter). A conductive track of the antenna may have a width within the range of 1 mm (millimeter) to 10 mm, preferably a constant width. The carrier substrate portion which carries the antenna may have a width which is more than the width of the conductive track of the antenna and which is preferably less than twice the width of the conductive track of the antenna.

In all cases the antenna may be a quarter-lambda antenna, having a length equal to or about equal to a quarter of the wave length of the transmission frequency. “About” may mean plus or minus 10 percent or plus or minus 5 percent less or more compared to the wave length of the transmission frequency. Dielectric characteristic and/or wavelength within material and/or a velocity factor (VF) (wave propagation speed, velocity of propagation VoP) may also be considered for determining the length of the antenna. However, other lengths of the antenna may also be considered to be appropriate.

The antenna may be arranged and configured for at least one of the following features:
reducing antenna directivity, e.g. by enhancing an isotropic radiation characteristic, e.g. a monopole antenna or a dipole antenna or another type of antenna that extends originally or usually along a straight line and/or within a plane may be arranged to form a ring in order to have a more isotropic characteristic compared to an anisotropic radiation characteristic in another conformation, e.g. in the linear extended conformation, and/or
transforming a linear polarization as given by the original underlying antenna geometry to a more elliptical/mixed polarization, e.g. mitigating a linear polarization by choosing an appropriate antenna and/or shape of antenna and/or arrangement of the antenna, and/or
being arranged between at least one distal electronic circuit and at least one proximal electronic circuit, e.g. in order to enhance/modify the radiation pattern.

The radiation characteristic and/or elliptical polarization may be influenced by two electrical circuits on each side of the antenna, e.g. a distal mainboard and a proximal LED (light emitting device) board.

Without being bound by theory, a comparably isotropic antenna and/or a more elliptical/circular polarized antenna may have the technical effect that a rotation and/or pivoting of the drug delivery device or of another pen like device does not have or has only minor influence to maximum transmission range and/or quality of transmission of data.

The arrangement, especially the body may comprise a longitudinal axis. A main portion of the antenna may extend around the longitudinal axis. An angular (circumferential) extension of the antenna may be at least one of the following values: 180, 200, 220, 240, 260, 280 degrees. The angular extension may be less that one of the following values: 360, 340, 320, 300 degrees. Alternatively the angular extension may be more than one of the following values: 360 degrees, 540, 720 degrees. The antenna may comprise less than 10 windings to give only one example. Winding or wrapping the antenna around the body may result in a low form factor of the overall device. Other technical effects may refer to the radiation characteristic and/or to the polarization characteristic of the antenna during transmission (sending) and/or receiving of electromagnetic radiation.

A main direction of extension of the antenna may be in the angular direction. The main direction of extension may relate to the overall extension of the antenna.

The angular extension of the antenna may be at least three times or at least five times the axial extension of the antenna. The antenna may be positioned at a predetermined axial position. The circumferential extension of the antenna may be at least three times or at least five times the axial extension of the antenna in an axial direction of the drug delivery device.

The antenna may be arranged at essentially the same or at the same radial distance from a longitudinal axis of the drug delivery device. “Essentially” may refer do deviations of maximum 5 percent relative to a maximal radial distance of the antenna. Thus, the antenna may be arranged at the same radial distance to a longitudinal axis of the drug delivery device for different or for all angular positions of the antenna. This may enable usage of a body comprising a circular cross section or an essentially circular cross section. However, the radial distance may also vary, e.g. if the body has a more elliptical cross section.

A winding axis of the antenna may be parallel to the longitudinal axis of the drug delivery device.

Without being bound by theory, rotation of the drug delivery device may have no or may have only minor influence to maximum transmission range and/or quality of transmission of data if an antenna with a comparably isotropic radiation characteristic is used.

The curvature of the antenna in the circumferential direction may be equal to or may essentially correspond to an inner curvature or to an outer curvature of the body, e.g. of a housing member or of a chassis, preferably of an inner chassis.

Alternatively, the antenna may be arranged along a helical path, e.g. the axial position of the antenna may vary with increasing length of the antenna. The resulting spiral may allow an adaption of the length of the antenna to a length of the body, for instance dependent on a pitch angle or on a pitch value. The pitch angle may be comparably low, e.g. less than 45 degrees or less than 30 degrees.

Alternatively, a main direction of extension of the antenna may be along the longitudinal axis of the drug delivery device. The axial extension of the antenna may be at least three times or at least five times the angular extension of the antenna. The antenna may be positioned at a predetermined angular position. Thus, a main portion of the antenna may extend in a direction which is essentially parallel or which is parallel to the longitudinal axis.

An angular (circumferential) extension of the antenna may be less that one of the following values: 180, 160, 140, 120, 100, 90, 80, 60, 40 or 20 degrees.

Without being bound by theory, also a comparably anisotropic radiation and/or linear polarization may be appropriate for some use cases. There may be other technical effects of the proposed arrangement, e.g. simple mechanical support for the antenna and/or easy manufacturability, etc.

The curvature of the antenna in the circumferential direction may be equal to or may be essentially correspond to an inner curvature or to an outer curvature of the body, e.g. of a housing member or of a chassis.

The antenna may be arranged at the same radial distance from a longitudinal axis of the drug delivery device for different or for all axial positions of the antenna.

The antenna may comprise a main portion and a connecting portion which are oriented in different directions. The connecting portion may be spatially and/or electrically arranged between the main portion and the electronic circuit. The main portion of the antenna may comprise at least 60 percent or at least 80 percent of the length of the antenna, especially of the length along a longitudinal axis of the antenna. If the antenna comprises only one conductive conductor the main portion may comprise at least 60 percent or at least 80 percent of the length of the electrically conductive conductor. However, it is also possible that the antenna comprises more than one conductive conductor. Both the main portion and the connecting portions may be used for sending and/or receiving the electromagnetic waves for communication purposes.

The connecting portion may be arranged at an angle relative to the main portion of the antenna. The angle may be in the range of 80 to 100 degrees, preferably 90 degrees. The angle may be measured, if the substrate is in an operation conformation and/or in a plane conformation. An angle of 90 degrees may allow a winding of the antenna in a plane perpendicular to a longitudinal axis of a housing portion and/or may ease assembly of the arrangement.

Furthermore, a main part of the circuit board (e.g. mainboard) may be arranged in a further plane which is perpendicular to a longitudinal axis of the body, e.g. of a chassis or of a housing member. This may enable a compact device and/or a low form factor, etc.

A first angle may be defined between the main portion of the antenna and the connecting portion of antenna. A second angle may be defined between the connecting portion and the mainboard of electronic circuit, e.g. an angle to the adjacent side of a rectangular mainboard or square mainboard or an angle with regard to radial direction or circular direction at connecting portion in case of a circular mainboard or of an elliptical mainboard. The sum of the first angle and of the second angle may be 180 degrees in order to realize an antenna that is wrapped around the circumference of a cylindrical body, especially within a plane.

The arrangement may comprise an electrical impedance matching network. The antenna may be electrically conductive connected to the electrical impedance matching network. The electrical impedance matching network may comprise passive electronic components, preferably discrete or “lumped” electronic components. Integration of these discrete elements, e.g. conductive tracks and/or capacitors (capacitance) and/or coils (inductance), into an integrated circuit may not be necessary. This may allow manufacturing costs to be reduced.

The impedance matching network may have any layout. A first capacitor may be connected to ground and to a first node of the electric circuit of the impedance matching network. A coil may be connected to the first node and to a second node of the electric circuit. A second capacitor may be connected to a second node of the circuit and to ground. The antenna may be connected to the first circuit node with or without an additional capacitor in series connection to antenna. The electronic circuit, e.g. an RF (radio frequency) input and/or RF output of the electronic circuit may be connected to the second node. Optionally, biasing may be performed on the second node of the circuit, e.g. via a further coil. However, differential operation of the antenna may be appropriate as well.

Single ended operation may be used with or without internal biasing. Power consumption may be low in both cases of single ended operation. The internal bias may enable further reduction in power consumption.

A first attachment region may be formed adjacent to a free end of the antenna on a carrier substrate of the antenna. The first attachment region may be configured to position the antenna relative to the body. The first attachment region may prevent a relative translational movement between the antenna and the body, preferably in at least two directions of a plane which are perpendicular with regard to each other or even in at least three directions which are perpendicular with regard to each other. The first attachment region may preferably be located at an extension of the longitudinal axis of a main portion of the antenna.

There may be a second attachment region adjacent to the other end of the main portion of the antenna, e.g. near the border line between the main portion of the antenna and of the connecting portion of the antenna. The second attachment region may also be configured to position the antenna relative to the body. The second attachment region may preferably be located at an extension of the longitudinal axis of the connecting portion of the antenna.

At least one of the respective attachment regions may comprise at least one of:
a neck region that has smaller width than an adjacent area of the attachment region and/or than the carrier substrate of the antenna between the antenna and the neck region, and
a through hole that extends through the attachment region.

Alternatively and or additionally, an adhesive may be used to attach the attachment region and therewith the antenna to the body.

The arrangement may comprise a guide structure to define the path of the antenna along the contour of the body. The main portion of the antenna may follow the guide structure. The guide structure may be a recess, e.g. a groove or a notch. Alternatively other guiding structures may be used, e.g. guiding pins. An outer groove on the body or an inner groove body may be used as guiding structures in order to define the position and/or the path of the antenna.

The body may comprise at least one attachment element that may be configured to interact with at least one attachment region of the arrangement in order to prevent relative movement of the antenna and of the body along a main extension direction of the antenna. Protrusions may be arranged on the body, e.g. within the guide structure mentioned above, preferably within a notch or groove. Alternatively, the antenna may have protrusions which interact with recesses on the body, e.g. with recesses within the guide structure mentioned above, preferably within the notch or groove on the body.

There may be an axial interference of protrusions on the body and of a recess on the carrier of the antenna. The protrusions may be arranged protruding cross a main extension direction of a main portion of the antenna.

At least one of the following attachment regions may be used:
two protrusions which are arranged opposite to each other within a groove, preferably protrusions which extend closer relative to each other within an upper part of the groove compared to a distance of the protrusions within a lower part of the groove, more preferably protrusions which are configured to interact with a neck region of the antenna arrangement, and
a clipping element which is clipped to the body in order to attach the antenna thereto, preferably comprising at least one protrusion which is configured to extend through a through hole within an attachment region of the antenna.

The clipping element may be an outer cover of a knob for dialing or setting a dose of the drug delivery device. Thus, the outer cover may fulfill multiple functions.

The disclosure relates also to a drug delivery device comprising the arrangement according to any one of the embodiments mentioned above. Thus, the same technical effects do apply. The drug delivery device may comprise at least one of or all of:
a medicament/drug container,
an optional medicament/drug container holding part, preferably made as a separate part of the drug delivery device compared to a main housing,
a piston rod which may be adapted to be inserted into the medicament container during injection, and
a drive spring which may be configured to drive the piston rod or a pushing element that is operatively coupled to the piston rod for manually driving the piston rod.

The arrangement may be an integral part of the drug delivery device. Thus, the arrangement and the drug delivery device may share the same housing and/or the same chassis arranged within an outer housing. Alternatively, the arrangement may be a module that is releasably or non-releasably connected to a main housing of the drug delivery device. “Releasably connected” may mean that both components may be released from each other without destruction of the drug delivery device and/or without using tools. Release of the module may allow multi-usage of the module together with several devices, e.g. with several drug delivery devices. This may have a huge environmental impact and may allow cost advantages.

A further aspect of the disclosure relates to a user interface module, preferably to a rotatable dose adjusting knob. The user interface module may comprise the arrangement according to any one of the embodiments mentioned above. Thus, the same technical effects do apply. The user interface module may comprise at least one of the following:
at least one sensor that is configured to detect a movement or a rotation of an element of the drug delivery device during injection of a medicament or drug and/or during adjustment of a dose of the medicament or drug to be injected, and
at least one mechanical interface that is configured to be connected to a drug delivery device.

The mechanical interface may comprise a clip connection, a threaded connection or another appropriate mechanical connection.

The user interface module may not comprise or may be free of a medicament/drug container and/or a piston rod and/or a drive spring. The user interface module may have a length of less than 10 cm (centimeters), less than 5 cm or less than 3 cm. However, a longer user interface module may also be used. The usage of the module may enable separate handling of mechanical assemblies and of electronic assemblies, for instance by different manufacturers of different industries.

The electronic circuit may comprise a processor and a memory. The memory (e.g. RAM (random access memory), ROM (read only memory), SSD (solid state device)) may store instructions which may be executed by the processor. During execution of the instructions an operation of dose recording may be performed. Data relating to the recorded doses may be transmitted (sent) via the antenna to an external data processing device or computer. Receiving of control messages via the antenna may be performed according to standard transmission protocols.

Bluetooth (may be a trademark) or another data transmission protocol may be used, e.g. Bluetooth low energy (BLE (may be a trademark)). Therefore, the electronic circuit may comprise a communication circuit. The communication circuit may be implemented by the processor.

As already mentioned above, the electronic circuit may comprise at least one sensor that allows the detection or measurement of amounts of a medicament/drug to be delivered or delivered. Examples of sensors are radiation sensors, e.g. optical sensors, accelerometer, pressure sensor, proximity sensors, sound sensor, e.g. ultrasonic, capacitive sensors, inductive sensors or resistive sensors. The sensor may enable a contact free or contactless detection. Alternatively, mechanical contact between the sensor and a further part may be necessary during detection, e.g. to a rotating part.

The user interface module may comprise at least one of the following:
an end cap, preferably comprising a light absorbing layer and/or a light conducting layer, e.g. appropriate to guide light of at least one user interface LED (light emitting device),
at least one power supply element, e.g. a battery or a rechargeable battery, preferably within a compartment of the user interface module, and
at least one light emitting diode (LED) as part of a user interface, preferably within a compartment of the user interface module or on the outside of the user interface module.

The user interface module may be manufactured as a separate part compared to a main housing of the drug delivery device. The user interface module may be an electronic module that is connected to a mechanical module, especially to a purely mechanical module, in order to complete the drug delivery device.

A further aspect of the disclosure relates to a method of manufacturing, preferably in the following sequence
a) providing a first body or alternatively a first body and a second body and providing a printed circuit board comprising a flexible antenna,
b) placing the printed circuit board within the first body or within a second body, and
c) wrapping the flexible antenna around the first body.

In the following, a set of aspects is disclosed. The aspects are numbered to facilitate referencing the features of one aspect in other aspects. The aspects form part of the disclosure of the present application and could be made subject to independent and/or dependent claims irrespective of what currently is claimed in the application and also independent of the references in brackets.
1. Arrangement (601) for a drug delivery device, comprising:
at least one electronic circuit (EC),
an antenna (501) for wireless communication, and
a body,
wherein the antenna (501) is operatively connected to the electronic circuit (EC),
wherein the antenna (501) is flexible, and
wherein the antenna (501) is adapted to follow a contour defined by the body,
wherein preferably the antenna (501) is arranged on a flexible carrier substrate (506, 508),
wherein preferably the flexible carrier substrate (506, 508) is part of a circuit board (2) of the electronic circuit (EC),
and wherein preferably the antenna (501) is wrapped around the body and the electronic circuit (EC) is arranged within the body.
2. Arrangement (601) according to aspect 1, wherein a curvature of the antenna (501) follows a curvature that is defined by the body.
3. Arrangement (601) according to aspect 1 or aspect 2, wherein the antenna (501) is arranged on a flexible carrier substrate (506, 508), and
wherein the flexible carrier substrate (506, 508) is part of a circuit board (2) of the electronic circuit (EC), preferably part of a multilayer circuit board (2).
4. Arrangement (601) according to any one of the preceding aspects, wherein the antenna (501) is a monopole antenna or a dipole antenna.
5. Arrangement (601) according to any one of the preceding aspects, especially according to aspect 4, wherein the antenna (501) is arranged and configured for at least one of the following features:
reducing antenna directivity, and
transforming a linear polarization to a more elliptical polarization and/or mixed polarization, and being arranged within a region (R1) between at least one distal electronic circuit at least one electronic circuit (EC) and at least one proximal electronic circuit at least one electronic circuit (EC).
6. Arrangement (601) according to any one of the aspects 1 to 5, wherein the arrangement (601) comprises a longitudinal axis (L),
wherein a main portion (502) of the antenna (501) extends around the longitudinal axis (L), and
wherein an angular extension of the antenna (501) is at least 180 degrees.
7. Arrangement (601) according to aspect 6, wherein a main direction of extension of the antenna (132b, 134b, 501) is in the angular direction (C).
8. Arrangement (601) according to any one of the preceding aspects,
wherein the arrangement (601) comprises a longitudinal axis (L),
wherein a main direction of extension of the antenna (132c, 134c) is along the longitudinal axis (L).
9. Arrangement (601) according to any one of the preceding aspects, wherein the antenna (501) comprises a main portion (502) and a connecting portion (504) which are oriented in different directions, and
wherein the connecting portion (504) is arranged between the main portion (502) and the electronic circuit (EC).
10. Arrangement (601) according to any one of the preceding aspects, comprising an electrical impedance matching network,
wherein the antenna (501) is connected to the electrical impedance matching network, and
wherein the electrical impedance matching network comprises at least two passive electronic components that are discrete electronic components.
11. Arrangement (601) according to any one of the preceding aspects, wherein a first attachment region (510a) is formed adjacent to a free end of the antenna (501) on a carrier substrate (506) of the antenna (501),
wherein the first attachment region (510a) is configured to position the antenna (501) relative to the body.
12. Arrangement (601) according to any one of the preceding aspects, wherein the arrangement comprises a guide structure to define the path of the antenna (501) along the contour of the body, preferably a recess, e.g. a groove or a notch.
13. Arrangement (601) according to any one of the preceding aspects, preferably according to aspect 12, wherein the body comprises at least one attachment element that is configured to interact with at least one attachment region (510a) of the arrangement (601) in order to prevent relative movement of the antenna (501) and the body along a main extension direction of the antenna (501).
14. Arrangement (601) according to aspect 3, wherein the antenna is wrapped around the body, preferably in order to mitigate directivity of the antenna (501), and the electronic circuit (EC) is arranged within the body.
15. Arrangement (601) according to aspect 3 and 9, wherein the connecting portion (504) is arranged at an angle relative to the main portion (502) of the antenna (501),
wherein the angle allows a winding of the antenna (501) in a plane perpendicular to a longitudinal axis (L) of the body, preferably in order to mitigate directivity of the antenna (501), and
wherein a main part of the circuit board (2) is arranged in a further plane which is perpendicular to the longitudinal axis (L) of the body.
16. Drug delivery device (100) comprising the arrangement (601) according to any one of the previous aspects, wherein the drug delivery device (100) comprises at least one of or all of:
   a medicament container,
   an optional medicament container holding part (101), preferably made as a separate part of the drug delivery device (100) compared to a main housing (102),
   a piston rod (104) which is adapted to be inserted into the medicament container during injection, and
   a drive spring which is configured to drive the piston rod (104) or a pushing element that is operatively coupled to the piston rod (104) for manually driving the piston rod (104).
17. User interface module (120), preferably rotatable dose adjusting knob (600), comprising the arrangement (601) according to any one of the aspects 1 to 15, wherein the user interface module (120) comprises at least one of the following:
   at least one sensor that is configured to detect a movement or a rotation of an element of the drug delivery device (100) during injection of a medicament (Dr) or during adjustment of a dose of a medicament (Dr) to be injected, and
   at least one mechanical interface that is configured to be connected to a drug delivery device (100)
18. Method of manufacturing an arrangement (601) according to any one of the aspects 1 to 15 or a drug delivery device (100) according to aspect 16 or a user interface module (120) according to aspect 17, preferably in the following sequence
   a) providing a first body and providing a printed circuit board (2) comprising a flexible antenna (51),
   b) placing the printed circuit board (2) within the first body, and
   c) wrapping the flexible antenna (501) around the first body, preferably in order to mitigate directivity of the antenna (501).

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this application it means the possibility of doing so as well as the actual technical implementation. The present concepts of the present disclosure will be described with respect to preferred embodiments below in a more specific context namely drug delivery devices, especially drug delivery devices for humans or animals. The disclosed concepts may also be applied, however, to other situations and/or arrangements as well, for instance for pen like or pencil like devices or for pens or pencils, especially electronic pens or pencils, e.g. Apple pen (may be a trade mark), which may be used to input handwriting into a computer device by writing on a touchscreen or on another sensitive display device.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale.

### BRIEF DESCRIPTION OF THE FIGURES

In the drawings the following is shown in:

FIG. 1 a drug delivery device comprising an electronic module,

FIG. 2 a drug delivery device according to a second embodiment comprising a circumferentially arranged antenna,

FIG. 3 a drug delivery device according to a second embodiment comprising an axially arranged antenna,

FIG. 4 a drug delivery device according to a second embodiment comprising an obliquely arranged antenna,

FIG. 5 a circuit board (flex-board) of a drug delivery device comprising a flexible antenna,

FIG. 6 a dose setting knob of a drug delivery device, and

FIG. 7 an electronic unit of a drug delivery device.

### DETAILED DESCRIPTION

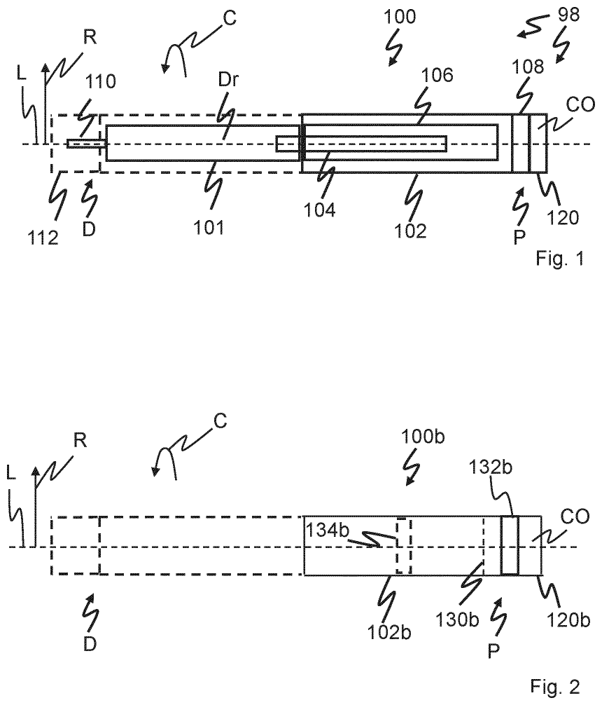

FIG. 1 illustrates a drug delivery device 100 that may comprise an optional container retaining member 101 and a main housing part 102. Container retaining member 101 may comprise a container comprising a drug Dr. Main housing part 102 may house or surround the container and/or container retaining member 101 completely or partially and may comprise further parts of the drug delivery device 100. Alternatively, the main housing part 102 may be connected to the container retaining member 101 but may not surround it and even may not surround a part of the container retaining member 101, see dashed line in FIG. 1.

Within the main housing part 102 the following components may be arranged:
a piston rod 104 that is adapted to move the piston that may be arranged within container retaining member 101,
a driving mechanism 106 for the piston rod 104. The driving mechanism 106 may comprise an energy storing element, for instance a spring that is loaded manually before each use. Alternatively, the energy storing element may be loaded for instance during assembling of drug delivery device 100. Alternatively, a manually driven driving mechanism may be used, e.g. without an energy storing element that is used to drive piston rod 104.
for instance, at a proximal end P, an actuating element may be used for the initiation of a movement of piston rod 104 into the container retaining member 101, whereby the driving mechanism 106 is used. Alternatively, an autoinjector device may be used that is actuated by an axial movement of a movable needle shroud (not shown). An actuating element or a dosing element may be used to dial the size or amount of a dose of drug Dr in some embodiments.
an optional cap 112 that may be attached to main housing part 102 or to another part of drug delivery device 100. Cap 112 may be an outer cap that may include a smaller inner cap which protects a needle 110 directly.

If drug delivery device 100 is not an autoinjector, a dial sleeve may be screwed out of main housing 102 and may be pressed by a user in order to move piston rod 104 (plunger) distally and to inject drug Dr.

Drug delivery device 100 may be a single use or a multiple use device.

Drug Dr may be dispensed from the container through needle 110 or through a nozzle that is connectable and/or connected to the distal end D of drug delivery device 100. Needle 110 may be changed before each use or may be used several times.

Drug delivery device 100 may comprise an electronic module 120 that is mechanically connected to a proximal end region P of drug delivery device 100. Electronic module 120 may be arranged for instance within an actuating element or at the proximal end P of drug delivery device 100 in the case of an autoinjector. Drug delivery device 100 is described below in more detail.

Electronic module 120 may be used not only for drug delivery device 100 but also for other drug delivery devices that are similar or identical to drug delivery device 100. Thus, electronic module 120 may be used multiple times with different drug delivery devices in different modular systems. Alternatively, electronic module 120 may only be used together with only one drug delivery device 100.

Furthermore, the diameter of drug delivery device 100 may be not increased by electronic module 120 promoting excellent handling of drug delivery device 100.

FIG. 1 illustrates a longitudinal axis L of drug delivery device 100. Longitudinal axis L may be a symmetry axis. Furthermore, a radial direction R is illustrated and a circumferential direction C. Reference may be made to a cylindrical coordinate system, i.e. each position may be defined by three coordinates: axial value (height, distance to zero plane), radial distance to axis and angle between current radial position and a plane that is defined as having angle zero.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes);

carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

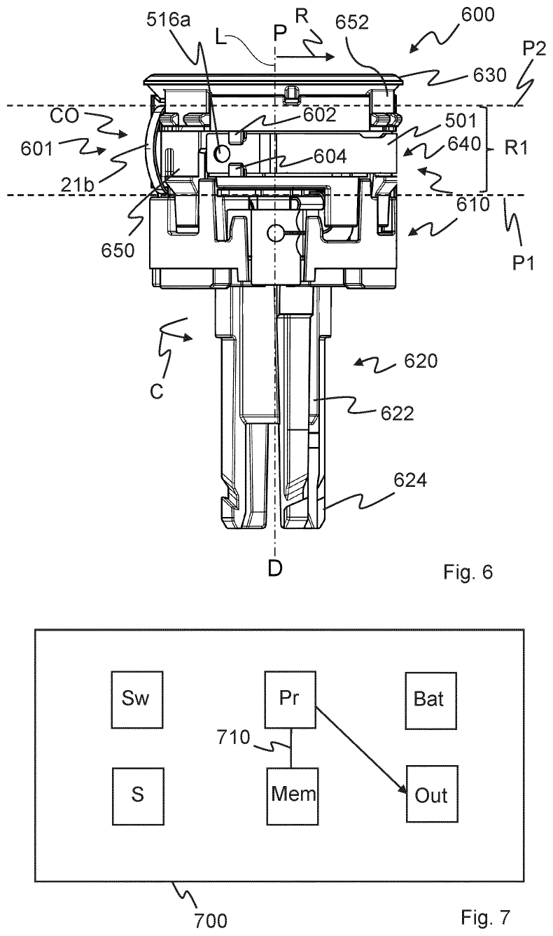

Drug delivery device 100 and all drug delivery devices 100b, 100c und 100d which are mentioned in the following may comprise an arrangement, especially an arrangement 600, see FIG. 6. The arrangement may comprise:
at least one electronic circuit EC,
an antenna 132b, 134b, 132c, 134c, 132d, 134d or 501 for wireless communication, and
a body, e.g. a housing of an electronic module 120, 120b, 120c or 120d, a main housing 102, 102b, 102c, 102d or a first housing portion 610.

The antenna 132b, 134b, 132c, 134c, 132d, 134d or 501 may be operatively connected to the electronic circuit EC. The antenna 132b, 134b, 132c, 134c, 132d, 134d or 501 may be flexible or rigid in some embodiments, see for instance embodiment of FIG. 4. The antenna 132b, 134b, 132c, 134c, 132d, 134d or 501 may be adapted to follow a contour defined by the body, e.g. e.g. a housing of an electronic module 120, 120b, 120c or 120d, a main housing 102, 102b, 102c, 102d or a first housing portion 610.

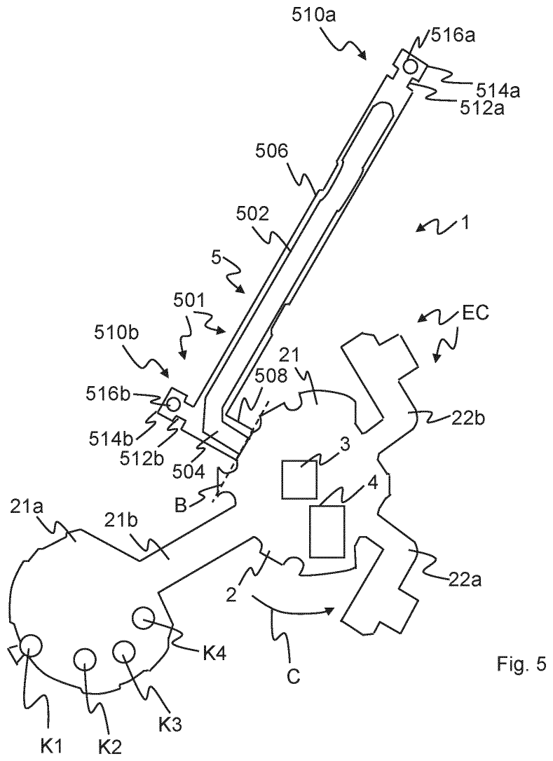

FIG. 2 illustrates a drug delivery device 100b according to a second embodiment comprising a circumferentially arranged antenna 132b. Drug delivery device 100b may be a drug delivery device 100 as described above with reference to FIG. 1. Drug delivery device 100b may comprise an electronic module 120b. Module 120b may comprise a compartment CO. Compartment CO may comprise an electronic circuit, see for instance electronic circuit EC as illustrated in FIG. 5 or another appropriate electronic circuit. The electronic circuit may comprise an antenna 132b that may be wrapped along the circumference of module 120b or around at least a part of the circumference. Module 120b may be a separate dose dialing knob 130b, e.g. as part of a user interface. Alternatively, module 120b may be an integral part of drug delivery device 100b.

If dose dialing knob 130b is used to dial a dose a plunger may extend proximally out of drug delivery device 100b. A user may press dose dialing knob 130b or a further button arranged at the proximal end P of dose dialing knob 130b in order to inject medicament/drug Dr. Details are explained below with reference to FIG. 6, e.g. dial knob 600 may be used as electronic module 120.

Alternatively, drug delivery device 100b may comprise an antenna 134b that is wrapped around a main housing 102b which may correspond to housing 102.

Both antennas 132b and 134b may be arranged within a plane that is perpendicular relative to longitudinal axis L of drug delivery device 100b.

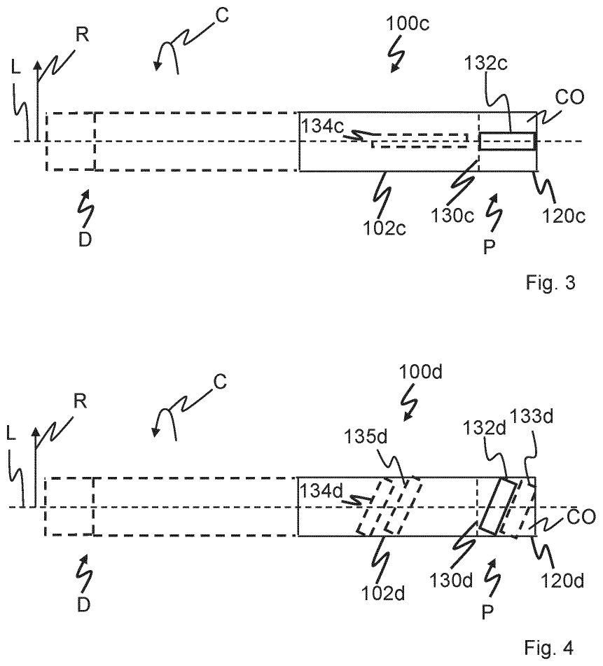

FIG. 3 illustrates a drug delivery device 100c according to a third embodiment comprising an axially arranged antenna 132c. Drug delivery device 100c may be a drug delivery device 100 as described above with reference to FIG. 1. Drug delivery device 100c may comprise an electronic module 120c. Module 120c may comprise a compartment CO. Compartment CO may comprise an electronic circuit, see for instance electronic circuit EC as illustrated in FIG. 5 or another appropriate electronic circuit. The electronic circuit may comprise an antenna 132c that may be arranged parallel to the longitudinal axis L of drug delivery device 100c at an outer surface or at an inner surface of module 120c. Module 120c may be a separate dose dialing knob 130c, e.g. as part of a user interface. Alternatively, module 120c may be an integral part of drug delivery device 100c.

If dose dialing knob 130c is used to dial a dose a plunger may extend proximally out of drug delivery device 100c. A user may press dose dialing knob 130c or a further button arranged at the proximal end P of dose dialing knob 130c in order to inject medicament/drug Dr. Module 120c may be similar to dial knob 600 illustrated in FIG. 6. The difference is that antenna 132c is arranged differently if compared with antenna 501. Antenna 132c may be arranged only on one side of drug delivery device 100c. Alternatively, antenna 132c may extend via a proximal end plate of module 130c and further on the other side of module 132c.

Alternatively, drug delivery device 100c may comprise an antenna 134c that extends parallel to longitudinal axis L of drug delivery device 100c on a surface of a main housing 102c which may correspond to housing 102. Antenna 134c may be arranged only on the illustrated side of drug delivery device 100c. Alternatively, antenna 134c may also extend through device 100c and also on the side of device 100c that is not illustrated in FIG. 3.

Both antennas 132c and 134c may be arranged within a plane that includes longitudinal axis L of drug delivery device 100c.

Thus, a main direction of extension of antenna 132c and 134c is along the longitudinal axis L.

FIG. 4 illustrates a drug delivery device 10d according to a fourth embodiment comprising an obliquely arranged antenna 132c. Drug delivery device 100d may be a drug delivery device 100 as described above with reference to FIG. 1. Drug delivery device 100d may comprise an electronic module 120d. Module 120d may comprise a compartment CO. Compartment CO may comprise an electronic circuit, see for instance electronic circuit EC as illustrated in FIG. 5 or another appropriate electronic circuit. The electronic circuit may comprise an antenna 132d that may be obliquely arranged relative to the longitudinal axis L of drug delivery device 100d at an outer surface or at an inner surface of module 120d. Module 120d may be a separate dose dialing knob 130d, e.g. as part of a user interface. Alternatively, module 120d may be an integral part of drug delivery device 100d.

If dose dialing knob 130d is used to dial a dose a plunger may extend proximally out of drug delivery device 100d. A user may press dose dialing knob 130d or a further button arranged at the proximal end P of dose dialing knob 130d in order to inject medicament/drug Dr. Module 120d may be similar to dial knob 600 illustrated in FIG. 6. The difference may be that antenna 132d is arranged differently if compared with antenna 501. Antenna 132d may be arranged within a plane that includes an acute angle with longitudinal axis L of drug delivery device 100d, e.g. antenna 132d may extend diagonal to longitudinal axis L.

Alternatively, drug delivery device 100d may comprise an antenna 134d that extends diagonally or obliquely to longitudinal axis L of drug delivery device 100d on a surface of a main housing 102d which may correspond to housing 102.

Both antennas 132d and 134d may be arranged within a plane that is arranged at an angle relative to longitudinal axis L of drug delivery device 100d. The angle may be in the range of 30 to 60 degrees, e.g. 45 degrees.

Thus, a main direction of extension of antenna 132d and 134d is along both the longitudinal axis L and the circumferential direction C.

There may be more than one winding of antenna 132d, see further winding 133d. Correspondingly, there may be more than one winding of antenna 134d, see further winding 135d.

FIG. 5 illustrates an electronic component 1. Electronic component 1 may comprise a circuit board 2, preferably a circuit board that is partially or completely realized as a flexible board (flex-board). Electronic component 1 may further comprise electronic elements mounted onto circuit board 2, e.g. a processor 3 (e.g. integrated circuit, IC) and/or a clock 4 (timer circuit, e.g. integrated circuit IC). Clock 4 may be used to trace the time of dose setting and/or dose injection events.

Circuit board 2 may have a central mainboard 21. Other parts of the circuit board are mentioned below and may be bendable and/or deflectable relative to mainboard 21. Thus, an antenna 501 may be “printed” or produced on circuit board 2. Antenna 501 may be flexible as is also mentioned below in more detail. Antenna 501 is also illustrated in FIG. 6 in its deflected conformation.

The electronic elements and the circuit board 2 may form an electronic circuit EC which may fulfill functions for detecting or measuring the amount of a dose of medicament/drug D set or delivered. Furthermore, electronic circuit EC may communicate dose related data and/or other relevant data via antenna 501 to an external device, e.g. to an electronic watch and/or to a computer device.

In addition to mainboard 21, circuit board 2 may comprise at least one of:
antenna section 5 that is described in more detail below,
an LED (light emitting diode) section 21a comprising at least one user interface LED or at least two user interface LEDs and for instance several contacts K1 to K4,
a connection section 21b which connects mainboard 21 and LED section 21a electrically, and
at least one or at least two sensor section(s) 22a, 22b which may carry at least one sensor or at least two sensors for measuring or detecting dose related data.

Printed circuit board (PCB) 2 may comprise only one carrier substrate layer or multiple carrier substrate layers, e.g. four layers of carrier substrates. Each carrier substrate side may be covered at least partially with conductive tracks and/or conductive areas. Isolating layers may be arranged between adjacent layers comprising conductive tracks and/or electrically conductive areas. Each carrier substrate may be a single-sided carrier substrate covered with conductive tracks and/or conductive areas only on one side or a two-sided carrier substrate that is covered with conductive tracks and/or conductive areas on both main sides.

Vias may connect conductive tracks and/or areas on different sides of a two-sided carrier substrate or connect conductive tracks and/or areas carried by different carrier substrates. The vias may extend through all carrier substrates or only through one or some of the substrate(s) but not through all carrier substrates at a specific location.

An antenna 501 may be formed only by one conductive track on one of the carrier layer(s), for instance in order to have an increased flexibility. However, other carrier substrate layers may also be present along antenna 501. Alternatively only one carrier substrate layer may be used for antenna 501, e.g. the other substrate carrier layers may not be present along antenna 501.

Antenna 501 may be a monopole antenna that has only one electrical connection to electronic circuit EC. Alternatively, a dipole antenna or another antenna may be used. Antenna 501 comprises a main portion 502 of antenna 501 and a connecting portion 504 of antenna 501. Main portion 502 may be carried by a main carrier substrate 506. Connecting portion 504 may be carried by a connecting carrier substrate 508. There may be a deflecting axis B between connecting carrier substrate 508 and mainboard 21. Antenna 501 may be deflected or be bent by for instance 90 degrees out of the plane which is illustrated in FIG. 5. Thus, antenna 501 may be arranged between a plane (see plane P1 in FIG. 6) in which mainboard 21 is arranged and a plane into which LED section 21a may be deflected, see plane P2 in FIG. 6.

Furthermore, antenna 501 may be deflected to form a closed loop or an almost closed loop, see also FIG. 6.

Main carrier substrate 506 may comprise on its free end an attachment region 510a.

Attachment region 510a may comprise:
a neck region 512a which has a minimum width of the antenna section 5,
a rectangular region 514a at the side of the neck region 512a which is farther away from antenna 501 than the other side of the neck region 512a, and
a through-hole 516a which is arranged within rectangular region 514a.

Connecting carrier substrate 508 may comprise on its end that is not connected to the mainboard 21 an attachment region 510b. Attachment region 510b may comprise:
a neck region 512b which has a minimum width of the antenna section 5,
a rectangular region 514b at the side of the neck region 512b which is farther away from antenna 501 than the other side of the neck region 512b, and
a through-hole 516b which is arranged within rectangular region 514b.

Thus, the first attachment region 510a may be formed adjacent to a free end of the antenna 501 on a carrier substrate 506 of the antenna 501. The first attachment region 510a may be configured to position the antenna 501 relative to the body, e.g. relative to first housing portion 610, see FIG. 6. Both attachment regions 510a, 510 may be configured to interfere with corresponding attachment elements that are described below in more detail, see for instance protrusions 602 and 604 illustrated in FIG. 6.

Electronic circuit EC may comprise an electrical impedance matching network. Antenna 501 may be connected to the electrical impedance matching network. The electrical impedance matching network may comprise passive electronic components that are for instance discrete or “lumped” electronic components. Details of the electrical impedance network are described in the introductory part of the description as well as technical effects thereof.

FIG. 5 illustrates recesses 520 and 522 on the carrier substrate 506 of the main portion 502 of antenna 501. Recess 520 is arranged on the left side of antenna 501 as illustrated in FIG. 5. Recess 522 is arranged on the right side of antenna 501 as illustrated in FIG. 5. Recess 520 is arranged closer relative to attachment region 510a than recess 522. There may be an optional slight change of the longitudinal direction of antenna 501 at recess 520. However, such a slight change of direction may also be made at recess 522 (not shown). Optional recesses 520 and 522 may have a function during manufacturing of circuit board 2 or other functions. Alternatively, no recesses 520 and 522 may be used.

FIG. 6 illustrates a dose setting knob 600 of a drug delivery device, for instance of one of the drug delivery devices 100 to 100d, especially 100b. Dose setting knob 600 may comprise:
an arrangement 601 comprising a body (610), antenna 501 and electronic circuit EC (not illustrated in FIG. 6,
a first housing member 610 (body),
an attachment portion 620,
a module cap 630,
a circumferential groove 640, and
attachment elements 650, 652, etc.

First housing portion 610 may be essentially cylindrical. First housing portion 610 may surround a compartment CO. Compartment CO may comprise electronic circuit EC, e.g. mainboard 21 arranged within a plane P1, LED section 21a arranged within a plane P2. Connecting section 21b is illustrated on the left side of dose setting knob 600.

Antenna 501 may be arranged within groove 640. Groove 640 may have a rectangular cross section, e.g. square or non-square. Two protrusions 602, 604 may be arranged within groove 640 interacting with neck region 512a of attachment region 510a.

At the back side of dose setting knob 600 there may be a vertical groove (not illustrated) which extends parallel to longitudinal axis L of dose setting knob 600. At the proximal end P or at the proximal portion of the vertical groove there may be two further protrusions which are similar to protrusions 602 and 604 and which may interact with neck region 512b of attachment region 510b.

Attachment portion 620 may form a mechanical interface for mounting the dose setting knob 600 to the proximal end of a drug delivery device 100 to 100d. Attachment portion 620 may comprise:
a central pin 622, e.g. a cylindrical pin,
at least one attachment arm 624, e.g. four arms as illustrated or less or more than four arms, more preferably each arm may comprise a hook at its distal end D in order to prevent release of dose setting knob 600 from drug delivery device 100 to 100d.

The attachment portion may comprise a thread according to an alternative embodiment. A thread may be used to establish a releasable connection between dose setting knob 600 and drug delivery device 100 to 100d.

Attachment elements 650, 652 may be used to fasten a cylindrical outer cover (not illustrated) of dose setting knob 600 onto dose setting knob 600. However, other fastening solutions may be used as well, e.g. gluing using an adhesive. The cylindrical outer cover may comprise pins which interfere with through holes 516a of attachment region 510a and 516b of attachment region 510b respectively.

Thus, antenna 501 may be arranged in an intermediate region R1 between plane P1 (distal) and plane P2 (proximal). Mainboard 21 and LED section 21a of circuit board 2 may have a significant influence to the radiation characteristic of antenna 501. However, the influence of mainboard 21 and LED section 21a of circuit board 2 with regard to polarization may be only small or negligible.

Antenna 501 may be arranged and configured for at least one of the following features:
enhancing an isotropic radiation characteristic, and/or
enhancing an elliptical and/or circular polarization, and/or
being arranged between at least one distal electromagnetic shielding region and at least one proximal electromagnetic shielding region.

Arrangement 601 comprises a longitudinal axis L. A main portion 502 of antenna 501 extends around the longitudinal axis L of arrangement 601. An angular extension of antenna 501 is at least 180 degrees, e.g. between 270 degrees and 360 degrees. Thus, a main direction of extension of antenna 132b, 134b, 501 is in the angular direction C.

Arrangement 601 may comprise a guide structure, e.g. groove 640, to define the path of antenna 501 along the contour of the body, e.g. first housing member 610.

The body, e.g. first housing member 610, may comprise at least one attachment element that may be configured to interact with at least one attachment region 510a of arrangement 601, e.g. with at least one attachment region 510a of antenna 501, in order to prevent relative movement, e.g. translational movement, of antenna 501 and body e.g. first housing member 610, along a main extension direction of antenna 501. Protrusions 602 and 604 are an example for such attachment elements.

FIG. 7 illustrates schematically an electronic unit 700. Electronic unit 700 may comprise:
at least one processor Pr, e.g. processor 3, or another control unit,
a memory Mem, for instance volatile and/or nonvolatile storing memory,
a battery Bat or a rechargeable battery/accumulator or any other electrical power source,
an output device Out, for instance a sending unit, for instance for communication with a smartphone or other computer device, and/or at least one user interface LED,
an optional input device In, for instance for communication with a smartphone or other computer device,
an optional switch Sw, and
at least one sensor S or at least two sensors, preferably optical sensor(s), preferably a sensor that is able to detect and/or to measure dose related parameters, e.g. amount of dose set and/or amount of dose injected.

Further parts may be comprised in electronic unit 700 that are not shown.

Processor Pr may be a microcontroller or microprocessor that performs instructions of a program which is stored in memory M. Alternatively, an FPGA (Field Programmable Gate Array), ASIC (Application Specific Integrated Circuit), PLA (Programmable Logic Array), PLD (Programmable Logic Device) or another appropriate circuitry may be used to implement a finite state machine that does not perform instructions of a program.

Spoken with other words, a design of an arrangement of a flexible antenna in a pen injection system is described for example. This disclosure describes a design of a flexible antenna in a pen injection device. The device may contain a single printed circuit board assembly (PCBA) comprised of flexible and/or rigid regions which is variously wrapped and folded into position within the device's outer body and/or inner body. The device may be capable of communicating with other external devices, for which an antenna may be required.

Without being bound by theory, due to the nature of pen injection devices, there may be no single defined orientation for operation or holding by the user. This may mean that a directional or anisotropic antenna may not be convenient when trying to communicate with another device. As such, it may be beneficial to aim for an isotropic characteristic. This may be achieved in an injection device by placing the antenna on a flexible region of the PCBA and wrapping it around a chassis (body) or housing component (body), e.g. a cylindrical chassis component.

The embodiments in this document are illustrated with an injection device, but are also applicable to many other devices, including disposable devices. In the case of disposable devices, e.g. disposable devices that are used only once, it is preferred to use a modular system enabling reuse of the electronic module.

This document does not include a full description of the device mechanism itself. For details of the operation of the device mechanism see all other applications of the applicant which are incorporated by reference herewith for all purposes.

The device, e.g. a Sanofi® device or a device of another manufacturer, may contain a single printed circuit board assembly (PCBA) comprised of flexible and/or rigid regions which may be variously wrapped and folded into position within the device's body, e.g. within the device's outer body. This may have the advantage that no additional connectors are needed to connect the electronic components. However, alternatively, it is of course possible to use at least two PCBs which are connected by connecting elements, preferably by flexible connecting elements.

The device may be capable of communicating with other external devices, for which an antenna is required or has advantages. In the case of an embodiment, this may be via Bluetooth (Special Interest Group), but other wireless communication technologies may be used. Other technologies are for instance, but not limited to, radio frequency RF, WiFi or WLAN (Wireless Local Area Network), see IEEE (Institute of Electrical and Electronics Engineers) 802.11 and/or IEEE 802.11 followed by a letter a to x where x is defined by the newest available standards, Zigbee of the Zigbee-Alliance, e.g. IEEE 802.15.4, Z-Wave of the Z-Wave Alliance, NFC (near field communication), etc.), RFID (radio frequency identification), etc.

Without being bound by theory, due to the nature of pen injection devices, there may be no single defined orientation for operation or holding by the user. For instance, one user may grip the device at the end and wrap their hand around it, whilst another may hold the device between thumb and forefinger, or some other way. This may mean that a directional or anisotropic antenna may not be convenient when trying to communicate with another device, as the range of transmission to another device such as a smartphone would vary between users. Furthermore, if the antenna effectively rotates, e.g. relative to another component (e.g. the outer body) during use, a user may be able to discern a variation in range between uses. As such, it may be beneficial to aim for an isotropic characteristic for the wireless communications. This may be achieved in the proposed injection device by placing the antenna on a flexible region of the PCBA and wrapping it around a chassis component (body), e.g. a cylindrical chassis component. The wrap may be close to 360° around the circumference producing a near isotropic characteristic. “Isotropic” may mean “omnidirectional”, i.e. with low directivity. The radiation may be essentially homogenous in all spatial directions. Wrapping the antenna in this manner within the structure may give it a 3D (three-dimensional) form generating a mixed polarization and/or making the radiation polarization more elliptically polarized, bringing for instance the advantage of less directivity from the user's point of view.

However, in other embodiments an isotropic antenna or a more isotropic antenna may be preferred, e.g. in embodiments in which the antenna extends essentially parallel to the longitudinal axis of the pen device, for instance of a drug delivery device.

This embodiment may utilize a quarter wave monopole antenna wrapped approximately 360° around the inner assembly. This may be different from a loop antenna which may have two different connecting nodes.

Other variants of antenna and wrapping could also be employed, for example, a dipole configuration with its antenna sections wrapping in opposite directions, or a monopole antenna wrapping more, or less, than 360°, etc. Further the wrapping may not necessarily need to be around the shortest circumference:
it may spiral around the axial symmetry of the device, or wrap across a diameter, or
it might wrap/follow a direction parallel with the device's axial symmetry, in this case it is possible to use a flexible antenna or a rigid antenna, or
it could be wrapped in some other manner that achieves a 3D shape within the confines of the assembly, or,
indeed any combination of these arrangements.

Typically, the antenna arrangement will be a compromise between complexity, maintaining for instance a reasonable omnidirectional performance, and efficiency or other appropriate criteria.

The dimensions and shape of the antenna conductor within the antenna structure may be designed to optimize the radio frequency efficiency and to meet requirements of the wireless communications system employed; in this case e.g. Bluetooth. These may deviate from ideal in order to better accommodate real world conditions. For example the antenna length may be different from that setup for free-space to account for the proximity of dielectric material such as a user's hand, table surface, etc. Further, the properties of the structure around which, and within which the antenna is wrapped or otherwise arranged may be also considered.

A quarter-wave monopole antenna in free space for the 2.4 GHZ (gigahertz) band used by Bluetooth may have a typical length of 31 mm (millimeter); when “printed” or produced on FR4 (fire resist class 4) PCB, that length is usually in the range of 20 mm to 25 mm. In this embodiment the length is close to 28 mm.

Further spoken with other words, it is also possible to extend the idea to more complex types of antennas compared to monopole or dipole antennas. Thus, the idea may be extended for instance to a Yagi antenna or to a Yagi-Uda antenna, i.e. antennas comprising a dipole and at least one, at least two, at least three or more than three directors and/or at least one, at least two, at least three or more than three reflectors. The dipole antenna and/or the director(s) and/or the reflector(s) may be arranged parallel to each other. To give a further example, a fractal antenna may be used, e.g. an antenna comprising structures comprising self-similarity. The principle may be summarized as mitigating the directivity by deflecting or bending the original or usual shape of the antenna, e.g. bending it to a more 2D (two-dimensional) or 3D (three-dimensional shape).

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. The embodiments mentioned in the first part of the description may be combined with each other. The embodiments of the description of figures may also be combined with each other. Further, it is possible to combine embodiments mentioned in the first part of the description with examples of the second part of the description which relates to FIGS. 1 to 7.

### REFERENCE NUMERALS

1 electronic component
2 printed circuit board PCB
3 processor
4 clock
5 antenna section
21 mainboard section
21a LED section
21b connection section
22a, 22b sensor section
100, 100b, c, d drug delivery device
101 container retaining element
102, 102b, 102c, 102d main housing part
104 piston rod
106 driving mechanism
110 needle
112 cap
P proximal
D distal
120 electronic module
CO compartment
L longitudinal axis
C circumferential direction
R radial direction
130b, 130c, 130d dialing knob
132b, 132c, 132d antenna on dialing knob
133d further winding
134b, 134c, 134d antenna on second housing portion
135d further winding
EC electronic circuit
501 antenna
502 main portion of antenna
504 connecting portion of antenna
506 main carrier substrate
508 connecting carrier substrate
510a, 510b attachment region
512a, 512b neck region
514a, 514b rectangular region
516a, 516b through-hole
K1 to K4 contact region
520, 522 recess
B bending axis
600 dose setting knob
601 arrangement
602, 604 protrusion
610 first housing member
620 attachment portion
622 central pin
624 attachment arm
630 module cap
640 groove
650, 652 attachment element
P1, P2 plane
R1 intermediate region
700 electronic unit
710 internal bus
Pr processor
Mem memory
Bat battery
Out output device
Sw switch
S sensor

1.-18. (canceled)

19. An apparatus for a drug delivery device, the apparatus comprising:
at least one electronic circuit;
an antenna for wireless communication; and
a body,
wherein the antenna is operatively connected to the electronic circuit,
wherein the antenna is flexible, and
wherein the antenna is adapted to follow a contour defined by the body.

20. The apparatus of claim 19, wherein the antenna is arranged on a flexible carrier substrate,
wherein the flexible carrier substrate is part of a circuit board of the electronic circuit,
wherein the antenna comprises a main portion and a connecting portion which are oriented in different directions,
wherein the connecting portion is arranged between the main portion and the electronic circuit,
wherein the connecting portion is arranged at an angle relative to the main portion of the antenna, wherein the angle allows a winding of the antenna in a plane perpendicular to a longitudinal axis of the body, and
wherein a main part of the circuit board is arranged in a plane which is perpendicular to the longitudinal axis of the body.

21. The apparatus of claim 19, wherein a curvature of the antenna follows a curvature that is defined by the body.

22. The apparatus of claim 19, wherein the antenna is arranged on a flexible carrier substrate, and
wherein the flexible carrier substrate is part of a circuit board of the electronic circuit.

23. The apparatus of claim 22, wherein the antenna is wrapped around the body and the electronic circuit is arranged within the body.

24. The apparatus of claim 22, wherein the antenna comprises a main portion and a connecting portion which are oriented in different directions,
wherein the connecting portion is arranged at an angle relative to the main portion of the antenna,
wherein the angle allows a winding of the antenna in a plane perpendicular to a longitudinal axis of the body, and
wherein a main part of the circuit board is arranged in a further plane which is perpendicular to the longitudinal axis of the body.

25. The apparatus of claim 19, wherein the antenna is a monopole antenna or a dipole antenna.

26. The apparatus of claim 19, wherein the antenna is arranged and configured for at least one of the following features:
reducing antenna directivity,
transforming a linear polarization to a more elliptical polarization and/or mixed polarization, or
being arranged within a region between at least one distal electronic circuit and at least one proximal electronic circuit.

27. The apparatus of claim 19, wherein the apparatus comprises a longitudinal axis,
wherein a main portion of the antenna extends around the longitudinal axis, and
wherein an angular extension of the antenna is at least 180 degrees.

28. The arrangement of claim 27, wherein a main direction of extension of the antenna is in an angular direction.

29. The apparatus of claim 19,
wherein the apparatus comprises a longitudinal axis, and
wherein a main direction of extension of the antenna is along the longitudinal axis

30. The arrangement of claim 19, wherein the antenna comprises a main portion and a connecting portion which are oriented in different directions, and
wherein the connecting portion is arranged between the main portion and the electronic circuit.

31. The apparatus of claim 19, comprising:
an electrical impedance matching network,
wherein the antenna is connected to the electrical impedance matching network, and
wherein the electrical impedance matching network comprises at least two passive electronic components that are discrete electronic components.

32. The arrangement of claim 19, wherein a first attachment region is formed adjacent to a free end of the antenna on a carrier substrate of the antenna, and
wherein the first attachment region is configured to position the antenna relative to the body.

33. The arrangement of claim 19, wherein the apparatus comprises a guide structure to define a path of the antenna along the contour of the body.

34. The arrangement of claim 19, wherein the body comprises at least one attachment element that is configured to interact with at least one attachment region of the apparatus in order to prevent relative movement of the antenna and the body along a main extension direction of the antenna

35. A drug delivery device comprising:
an apparatus comprising:
at least one electronic circuit;
an antenna for wireless communication; and
a body,
wherein the antenna is operatively connected to the electronic circuit,
wherein the antenna is flexible, and
wherein the antenna is adapted to follow a contour defined by the body; and
at least one of:
a medicament container,
a medicament container holding part,
a piston rod which is adapted to be inserted into the medicament container during injection, or
a drive spring which is configured to drive the piston rod or a pushing element that is operatively coupled to the piston rod for manually driving the piston rod.

36. The drug delivery device of claim 35, wherein the medicament container holding part is made as a separate part of the drug delivery device compared to a main housing.

37. A user interface module comprising:
an apparatus for a drug delivery device, the apparatus comprising at least one electronic circuit, an antenna for wireless communication, and a body, wherein the antenna is operatively connected to the electronic circuit, the antenna is flexible, and the antenna is adapted to follow a contour defined by the body,
wherein the user interface module comprises at least one of the following:
at least one sensor that is configured to detect a movement or a rotation of an element of the drug delivery device during injection of a medicament or during adjustment of a dose of a medicament to be injected, or
at least one mechanical interface that is configured to be connected to the drug delivery device.

38. The user interface module of claim 37, wherein the user interface module is a rotatable dose adjusting knob.
